(19) United States
(12) Patent Application Publication
(10) Pub. No.: US 2009/0143666 A1
(43) Pub. Date: Jun. 4, 2009

(54) System And Method For Non-Contrast Agent MR Angiography

(76) Inventors: Robert R. Edelman, Highland Park, IL (US); Ioannis Koktzoglou, Rolling Meadows, IL (US)

Correspondence Address: QUARLES & BRADY LLP, 411 E. WISCONSIN AVENUE, SUITE 2040, MILWAUKEE, WI, 53202-4497, US

(21) Appl. No.: 12/257,066

(22) Filed: Oct. 23, 2008

Related U.S. Application Data
(60) Provisional application No. 60/991,002, filed on Nov. 29, 2007.

(51) Int. Cl. A61B 5/055

(52) U.S. Cl. 600/410

(57) ABSTRACT

A system and method for imaging a desired region of the circulatory system uses the subtraction of data from two acquisitions using substantially different RF pulses and/or pulse sequence timing parameters. In one or both data sets, the longitudinal magnetization of spins within a selected imaging volume has been altered by the application of one or more RF preparatory (prep) pulses. The prep is applied in such a way that subtraction eliminates signals from static background spins, such as fat, while maintaining the signal intensity of intravascular spins.

### CROSS-REFERENCE TO RELATED APPLICATIONS

This application is based on, incorporates herein by reference, and claims the benefit of provisional application Ser. No. 60/991,002, filed Nov. 29, 2007, and entitled “SYSTEM AND METHOD FOR NON-CONTRAST AGENT MR ANGIOGRAPHY.”

### STATEMENT REGARDING FEDERALLY SPONSORED RESEARCH

Not applicable.

### FIELD OF THE INVENTION

The invention relates to a system and method for performing magnetic resonance angiography (MRA) and, more particularly, to a system and method for performing MRA without the need of a contrast agent.

### BACKGROUND OF THE INVENTION

When a substance such as human tissue is subjected to a uniform magnetic field (polarizing field B₀), the individual magnetic moments of the nuclear spins in the tissue attempt to align with this polarizing field, but precess about it in random order at their characteristic Larmor frequency. Usually the nuclear spins are comprised of hydrogen atoms, but other NMR active nuclei are occasionally used. A net magnetic moment M_{z} is produced in the direction of the polarizing field, but the randomly oriented magnetic components in the perpendicular, or transverse, plane (x-y plane) cancel one another. If, however, the substance, or tissue, is subjected to a magnetic field (excitation field B₁; also referred to as the radiofrequency (RF) field) which is in the x-y plane and which is near the Larmor frequency, the net aligned moment, M_{z}, may be rotated, or “tipped” into the x-y plane to produce a net transverse magnetic moment Mₜ, which is rotating, or spinning, in the x-y plane at the Larmor frequency. The practical value of this phenomenon resides in the signal which is emitted by the excited spins after the excitation field B₁ is terminated. There are a wide variety of measurement sequences in which this nuclear magnetic resonance (“NMR”) phenomenon is exploited.

When utilizing these signals to produce images, magnetic field gradients (Gₓ, G_{y}, and G_{z}) are employed. Typically, the region to be imaged experiences a sequence of measurement cycles in which these gradients vary according to the particular localization method being used. The emitted MR signals are detected using a receiver coil. The MRI signals are then digitized and processed to reconstruct the image using one of many well-known reconstruction techniques.

The ability to depict anatomy and pathology using MRI is dependent on the contrast, or difference in signal intensity between the target and background tissue. In order to maximize contrast, it is necessary to suppress the signal intensities of the background tissues. For instance, small blood vessels are much better depicted by the technique of MRA when the signal intensities of fat and muscle (background tissues) are minimized.

Magnetic resonance angiography (MRA) uses the NMR phenomenon to produce images of the human vasculature. There are three main categories of techniques for achieving the desired contrast for the purpose of MR angiography. The first general category is typically referred to as contrast enhanced (CE) MRA. The second general category is time-of-flight (TOF) MRA. The third general category is phase contrast (PC) MRA.

To perform CE MRA, a contrast agent, such as gadolinium, is injected into the patient prior to the magnetic resonance (MR) angiogram to enhance the diagnostic capability of the MR angiogram. Contrast enhanced MRA attempts to acquire the central k-space views at the moment the bolus of contrast agent is flowing through the vasculature being imaged. Collection of the central lines of k-space during peak arterial enhancement is important to the success of a CE MRA exam. If the central lines of k-space are acquired prior to the arrival of contrast, severe image artifacts can limit the diagnostic information in the image. Alternatively, arterial images acquired after the passage of the peak arterial contrast are sometimes obscured by the enhancement of veins.

While CE MRA is a highly effective means for noninvasively evaluating suspected vascular disease, the technique suffers from several additional drawbacks. First, the contrast agent that must be administered to enhance the blood vessel carries a significant financial cost. Second, contrast agents such as gadolinium have recently been shown to be causative of an often catastrophic disorder called nephrogenic systemic fibrosis (NSF). Third, CE MRA does not provide hemodynamic information, so that it is not always feasible to determine if a stenosis is hemodynamically significant. Fourth, the signal-to-noise ratio (SNR) and, therefore, spatial resolution is limited by the need to acquire data quickly during the first pass of contrast agent through a target vessel.

The 3D time-of-flight (TOF) techniques were introduced in the 1980s and they have changed little over the last decade. The 3D TOF MRA techniques commonly used for cranial examinations and have not been replaced despite recent advances in time-resolved contrast-enhanced 3D MRA. An alternative technique known as pulsed arterial spin labeling (PASL) was first applied to image intracranial circulation years ago; however, image quality never approached that of 3D TOF and the method has had little clinical utility. Moreover, electrocardiographic (ECG) gating was required. The use of TOF MRA is generally limited to imaging of intracranial circulation, however, because of sensitivity to patient motion and flow artifacts.

Finally, phase contrast MRA is largely reserved for the measurement of flow velocities and imaging of veins. It requires a longer scan time and the operator must set a velocity-encoding sensitivity, which varies unpredictably depending on a variety of clinical factors.

The signal targeting with alternating radiofrequency (STAR) technique, developed by Edelman et al. more than a decade ago, involves the application of an inversion B₁ pulse to spins outside of a selected region to be imaged, and not to the imaged region itself. The technique relies on the subtraction of two images sets in which background tissues have been exposed to precisely the same RF pulses. The STAR technique is ideally suited for imaging blood vessels containing fast blood flow, such as arteries, and is not well suited for imaging of veins containing slow blood flow.

The flow-sensitive alternating inversion recovery (FAIR) technique, along with the related FAIR with extra radiofrequency pulse (FAIRER) technique, applies a spatially non-selective inversion in one acquisition, and a spatially selective inversion to a region in the other acquisition. As in the case of STAR, it relies on the subtraction of two images sets in which background tissues have been exposed to precisely the same RF pulses. The method is primarily used for functional imaging of the brain and has not been used for MR angiography. It relies on inflow of spins into the selected region and is not suitable for imaging of veins. Moreover, it is highly sensitive to magnetization transfer effects that can result in imperfect image subtraction.

### SUMMARY OF THE INVENTION

The present invention provides a method for producing an angiogram with a magnetic resonance imaging (MRI) system without the need for administering a contrast agent. Specifically, the method includes performing a preparatory pulse sequence that includes application of an RF pulse that alters the longitudinal magnetization of spins in a region of interest. After a predetermined time interval (TI), the method includes performing an image acquisition pulse sequence to acquire complex image data in which the NMR signals from blood is suppressed, the NMR signals from fat are substantially recovered, and the NMR signals from other tissues are reduced. The method then includes repeating the image acquisition pulse sequence to acquire complex image data in which the NMR signals from blood is recovered, the NMR signals from fat are substantially recovered, and the NMR signals from other tissues are reduced. The method further includes repeating the preparatory pulse sequence and the two image acquisition pulse sequences to form a first complex image data set and a second complex image data. Additionally, the method includes performing a subtraction of the first and second image data sets to produce an angiogram in which blood vessels have an enhanced brightness.

The present invention provides a technique for MRA based on the distinctive relation properties and flow characteristics of blood. The method, called hereinafter Signal Targeting using Alternative Radiofrequency and Flow-Independent Relaxation Enhancement (STARFIRE) does not involve the administration of a contrast agent. It can be used to image both veins and arteries together, arteries alone, or veins alone, without substantial signal contributions from other tissues.

It is also contemplated that the present invention may be utilized to acquire and seamlessly merge two or more MRI data sets with distinctly different properties. The following method utilizes the above-described ability to generate arterial contrast through the use of arterial spin labeling, the use of image subtraction to suppress background signal (while maintaining substantial signal intensity from the arteries), and the use a single-shot or multi-shot pulse sequence.

A first pulse sequence is utilized that is specifically constructed to be insensitive to magnetic field inhomogeneities. Specifically, regions in which the local magnetic field homogeneity falls beneath a predetermined threshold (e.g. near the skull base) are designated for acquisition using the first pulse sequence. In accordance with one embodiment, the first pulse sequence includes a spoiled gradient-echo pulse sequence.

A second pulse sequence is utilized that is specifically constructed to provide the maximum intravascular signal intensity, but is more sensitive to magnetic field inhomogeneities than the first pulse sequence. In particular, regions in which the local magnetic field homogeneity is above the threshold level are designated for acquisition using second pulse sequence. In accordance with one embodiment, the second pulse sequence may include a balanced steady-state free precession (SSFP) pulse sequence and, more particularly, a segmented 3D trueFISP read-out may be used.

Both acquisitions generate arterial contrast through the use of arterial spin labeling, image subtraction, and a single-shot or multi-shot pulse sequence. The data acquired using the first pulse sequence and the second pulse sequence can be run in multi-shot or single-shot configurations.

It is widely recognized that an SSFP pulse sequence offers the benefit of high, often maximal signal-to-noise ratio (SNR) for the blood vessels. However, it is highly sensitive to the presence of static magnetic field inhomogeneities, as typically occur near bone or air-containing structures (e.g. the base of the skull or paranasal sinuses). Intravascular spins passing through such an inhomogeneous region experience a loss of signal intensity and flow artifacts are observed in the SSFP images. The spoiled gradient-echo pulse sequence is relatively insensitive to these static magnetic field inhomogeneities. However, the SNR is substantially lower than with SSFP. Moreover, images acquired with a spoiled gradient-echo pulse sequence suffer from substantial saturation effects which result in a loss of vessel signal intensity as the blood passes inside of the 3D imaging volume and is exposed to multiple repetitions of the excitatory RF pulse.

### BRIEF DESCRIPTION OF THE DRAWINGS

FIG. 1 is a block diagram of an MRI system for use with the present invention;

FIG. 2 is a schematic representation of a transceiver system for use with the MRI system of FIG. 1;

FIG. 3 is a diagram of a pulse sequence for use with the MRI system of FIG. 1;

FIG. 3A is a diagram of a prep pulse sequence for use in a process such as set forth in FIG. 4;

FIG. 3B is a diagram of a prep pulse sequence for use in a process such as set forth in FIGS. 5 and 6;

FIG. 4 is a flow chart setting forth the steps for imaging veins within a desired region of the circulatory system in accordance with the present invention;

FIG. 5 is a flow chart setting forth the steps for imaging arteries within a desired region of the circulatory system in accordance with the present invention;

FIG. 6 is a flow chart setting forth the steps for imaging veins and arteries within a desired region of the circulatory system in accordance with the present invention;

FIG. 7 is a flow chart setting forth the steps for imaging a desired region of the circulatory system having areas that do not induce magnetic field inhomogeneities and areas that do induce magnetic field inhomogeneities; and

FIG. 8 is a flow chart setting forth the steps for creating a cineagiogram of a desired portion of the circulatory system without the use of a contrast agent.

### DETAILED DESCRIPTION OF THE INVENTION

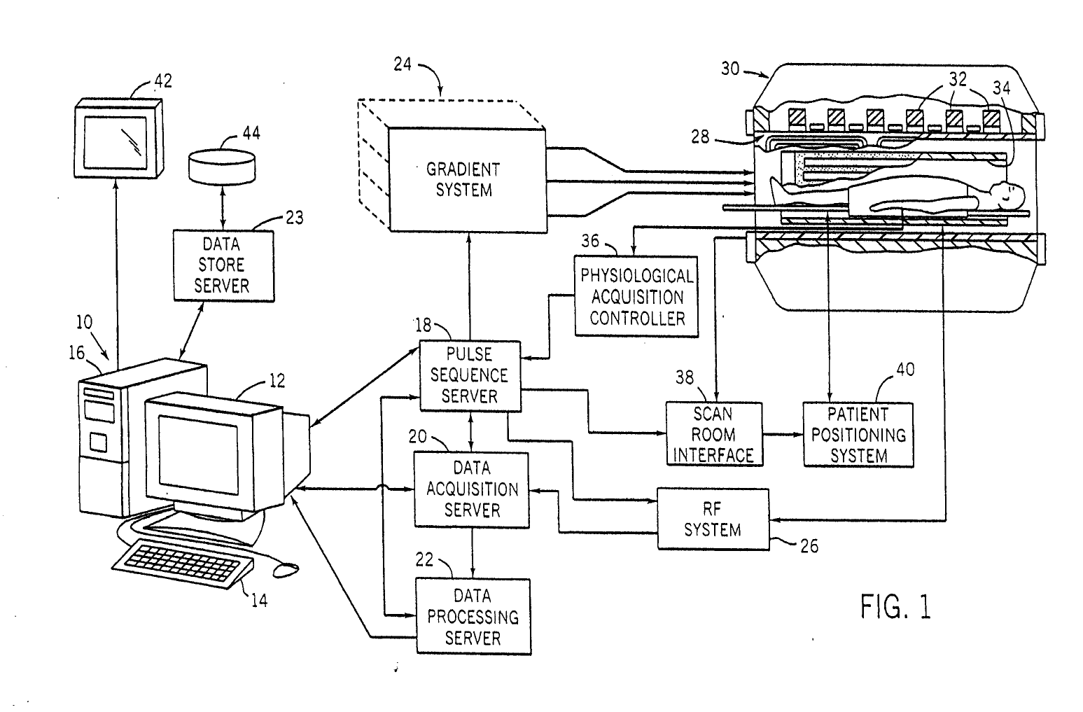

Referring particularly to FIG. 1, the preferred embodiment of the invention is employed in a MRI system. The MRI system includes a workstation 10 having a display 12 and a keyboard 14. The workstation 10 includes a processor 16 that is a commercially available programmable machine running a commercially available operating system. The workstation 10 provides the operator interface which enables scan prescriptions to be entered into the MRI system.

The workstation 10 is coupled to at least four servers, including a pulse sequence server 18, a data acquisition server 20, a data processing server 22, and a data store server 23. In one embodiment, the data store server 23 is performed by the workstation processor 16 and associated disc drive interface circuitry and the remaining three servers 18, 20, 22 are performed by separate processors mounted in a single enclosure and interconnected using a backplane bus. The pulse sequence server 18 employs a commercially available microprocessor and a commercially available communication controller. The data acquisition server 20 and data processing server 22 both employ commercially available microprocessors and the data processing server 22 further includes one or more array processors based on commercially available processors.

The workstation 10 and each processor for the servers 18, 20, 22 are connected to a serial communications network. This serial network conveys data that is downloaded to the servers 18, 20, 22 from the workstation 10 and conveys data that is communicated between the servers 18, 20, 22 and between the workstation 10 and the servers 18, 20, 22. In addition, a high speed data link is typically provided between the data processing server 22 and the workstation 10 in order to convey image data to the data store server 23.

The pulse sequence server 18 functions in response to program elements downloaded from the workstation 10 to operate a gradient system 24 and an RF system 26. Gradient waveforms necessary to perform the prescribed scan are produced and applied to the gradient system 24 that excites gradient coils in an assembly 28 to produce the magnetic field gradients Gₓ, G_{y}, and G_{z} used for position encoding NMR signals. The gradient coil assembly 28 forms part of a magnet assembly 30 which includes a polarizing magnet 32 and a whole-body RF coil 34.

The RF excitation waveforms are applied to the RF coil 34 by the RF system 26 to perform the prescribed magnetic resonance pulse sequence. Responsive NMR signals detected by the RF coil 34 are received by the RF system 26, amplified, demodulated, filtered, and digitized under direction of commands produced by the pulse sequence server 18. The RF system 26 includes an RF transmitter for producing a wide variety of RF pulses used in MR pulse sequences. The RF transmitter is responsive to the scan prescription and direction from the pulse sequence server 18 to produce RF pulses of the desired frequency, phase, and pulse amplitude waveform. The generated RF pulses may be applied to the whole body RF coil 34 or to one or more local coils or coil arrays.

The RF system 26 also includes one or more RF receiver channels. Each RF receiver channel includes an RF amplifier that amplifies the NMR signal received by the coil to which it is connected and a quadrature detector which detects and digitizes the in-phase (I) and quadrature (Q) components of the received NMR signal. The magnitude of the received NMR signal may thus be determined at any sampled point by the square root of the sum of the squares of the I and Q components.

The pulse sequence server 18 also optionally receives patient data from a physiological acquisition controller 36. The controller 36 receives signals from a number of different sensors connected to the patient, such as ECG signals from electrodes or respiratory signals from a bellows. Such signals are typically used by the pulse sequence server 18 to synchronize, or “gate”, the performance of the scan with the subject's respiration or heart beat.

The pulse sequence server 18 also connects to a scan room interface circuit 38 which receives signals from various sensors associated with the condition of the patient and the magnet system. It is also through the scan room interface circuit 38 that a patient positioning system 40 receives commands to move the patient to desired positions during the scan.

It should be apparent that the pulse sequence server 18 performs real-time control of MRI system elements during a scan. As a result, it is necessary that its hardware elements be operated with program instructions that are executed in a timely manner by run-time programs. The description components for a scan prescription are downloaded from the workstation 10 in the form of objects. The pulse sequence server 18 contains programs that receive these objects and converts them to objects that are employed by the run-time programs.

The digitized NMR signal samples produced by the RF system 26 are received by the data acquisition server 20. The data acquisition server 20 operates in response to description components downloaded from the workstation 10 to receive the real-time NMR data and provide buffer storage such that no data is lost by data overrun. In some scans, the data acquisition server 20 does little more than pass the acquired NMR data to the data processor server 22. However, in scans that require information derived from acquired NMR data to control the further performance of the scan, the data acquisition server 20 is programmed to produce such information and convey it to the pulse sequence server 18. For example, during prescans NMR data is acquired and used to calibrate the pulse sequence performed by the pulse sequence server 18. Also, navigator signals may be acquired during a scan and used to adjust RF or gradient system operating parameters or to control the view order in which k-space is sampled. Furthermore, the data acquisition server 20 may be employed to process NMR signals used to detect the arrival of contrast agent in an MRA scan. In all these examples the data acquisition server 20 acquires NMR data and processes it in real-time to produce information which is used to control the scan.

The data processing server 22 receives NMR data from the data acquisition server 20 and processes it in accordance with description components downloaded from the workstation 10. Such processing may include, for example, Fourier transformation of raw k-space NMR data to produce two or three-dimensional images, the application of filters to a reconstructed image, the performance of a backprojection image reconstruction of acquired NMR data, the calculation of functional MR images, the calculation of motion or flow images, and the like.

Images reconstructed by the data processing server 22 are conveyed back to the workstation 10 where they are stored. Real-time images are stored in a data base memory cache (not shown) from which they may be output to operator display 12 or a display 42 which is located near the magnet assembly 30 for use by attending physicians. Batch mode images or selected real time images are stored in a host database on disc storage 44. When such images have been reconstructed and transferred to storage, the data processing server 22 notifies the data store server 23 on the workstation 10. The workstation 10 may be used by an operator to archive the images, produce films, or send the images via a network to other facilities.

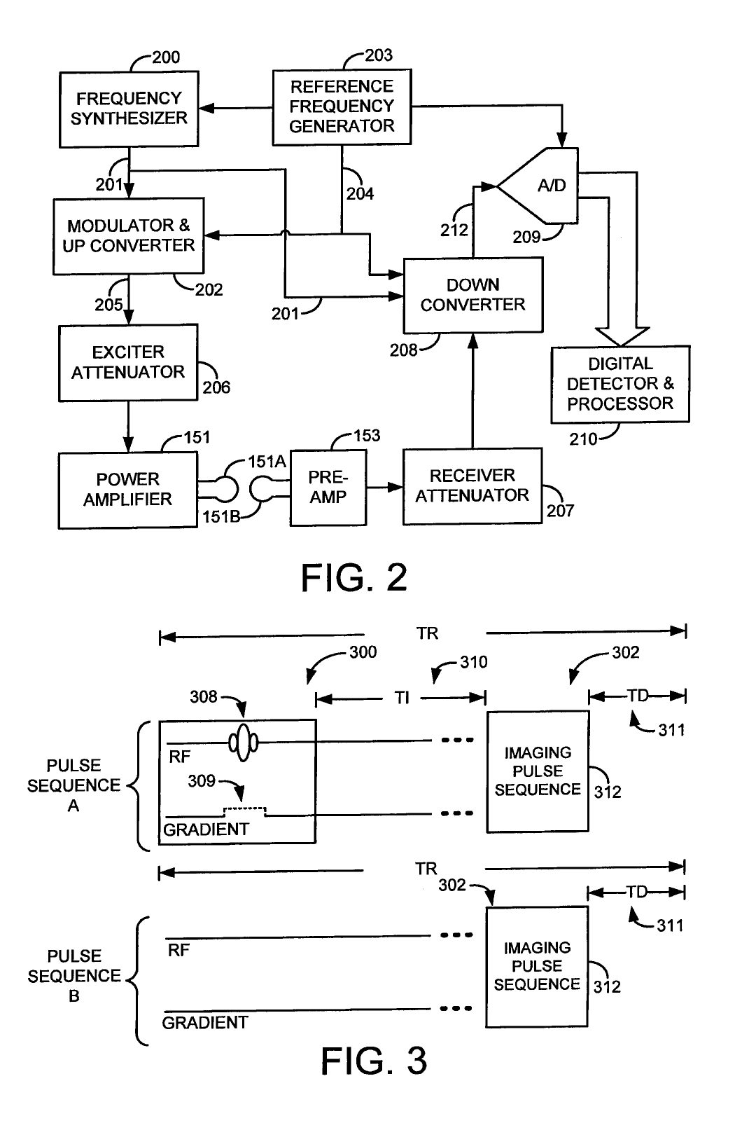

As shown in FIG. 1, the RF system 26 may be connected to the whole body RF coil 34, or as shown in FIG. 2, a transmitter section of the RF system 26 may connect to one RF coil 151A and its receiver section may connect to a separate RF receive coil 151 B. Often, the transmitter section is connected to the whole body RF coil 34 and each receiver section is connected to a separate local coil 151 B.

Referring particularly to FIG. 2, the RF system 26 includes a transmitter that produces a prescribed RF excitation field. The base, or carrier, frequency of this RF excitation field is produced under control of a frequency synthesizer 200 that receives a set of digital signals from the pulse sequence server 18. These digital signals indicate the frequency and phase of the RF carrier signal produced at an output 201. The RF carrier is applied to a modulator and up converter 202 where its amplitude is modulated in response to a signal R(t) also received from the pulse sequence server 18. The signal R(t) defines the envelope of the RF excitation pulse to be produced and is produced by sequentially reading out a series of stored digital values. These stored digital values may, be changed to enable any desired RF pulse envelope to be produced.

The magnitude of the RF excitation pulse produced at output 205 is attenuated by an exciter attenuator circuit 206 that receives a digital command from the pulse sequence server 18. The attenuated RF excitation pulses are applied to the power amplifier 151 that drives the RF coil 151A.

Referring still to FIG. 2, the signal produced by the subject is received by the receiver coil 152B and applied through a preamplifier 153 to the input of a receiver attenuator 207. The receiver attenuator 207 further amplifies the signal by an amount determined by a digital attenuation signal received from the pulse sequence server 18. The received signal is at or around the Larmor frequency, and this high frequency signal is down converted in a two step process by a down converter 208 that first mixes the NMR signal with the carrier signal on line 201 and then mixes the resulting difference signal with a reference signal on line 204. The down converted NMR signal is applied to the input of an analog-to-digital (A/D) converter 209 that samples and digitizes the analog signal and applies it to a digital detector and signal processor 210 to produce the I values and Q values corresponding to the received signal. As described above, the resulting stream of digitized I and Q values of the received signal are output to the data acquisition server 20 of FIG. 1. The reference signal, as well as the sampling signal applied to the A/D converter 209, is produced by a reference frequency generator 203.

As will be described, the present invention enables the subtraction of data from two acquisitions using substantially different RF pulses. In one data set, the longitudinal magnetization of spins within a selected imaging volume has been altered by the application of one or more RF pulses (referred to hereinafter as the prep). The prep is applied in such a way that subtraction eliminates signals from static background spins, such as fat, while maintaining the signal intensity of intravascular spins.

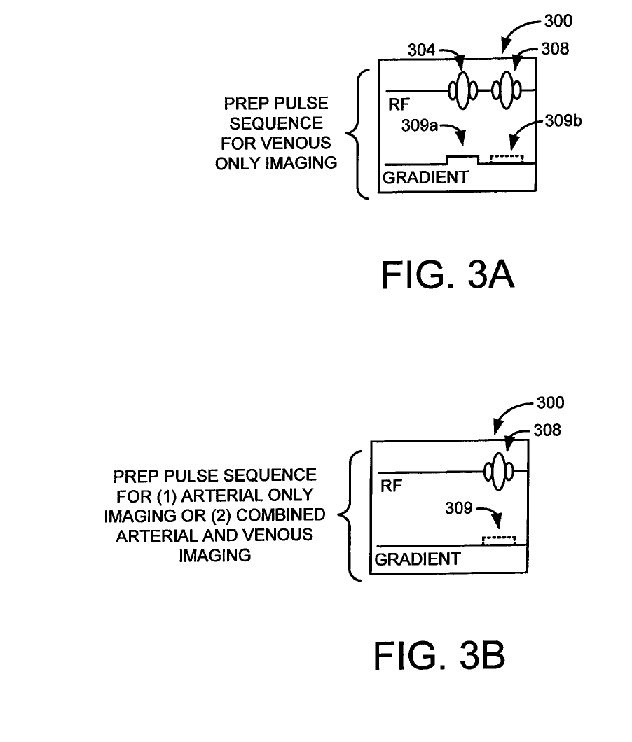

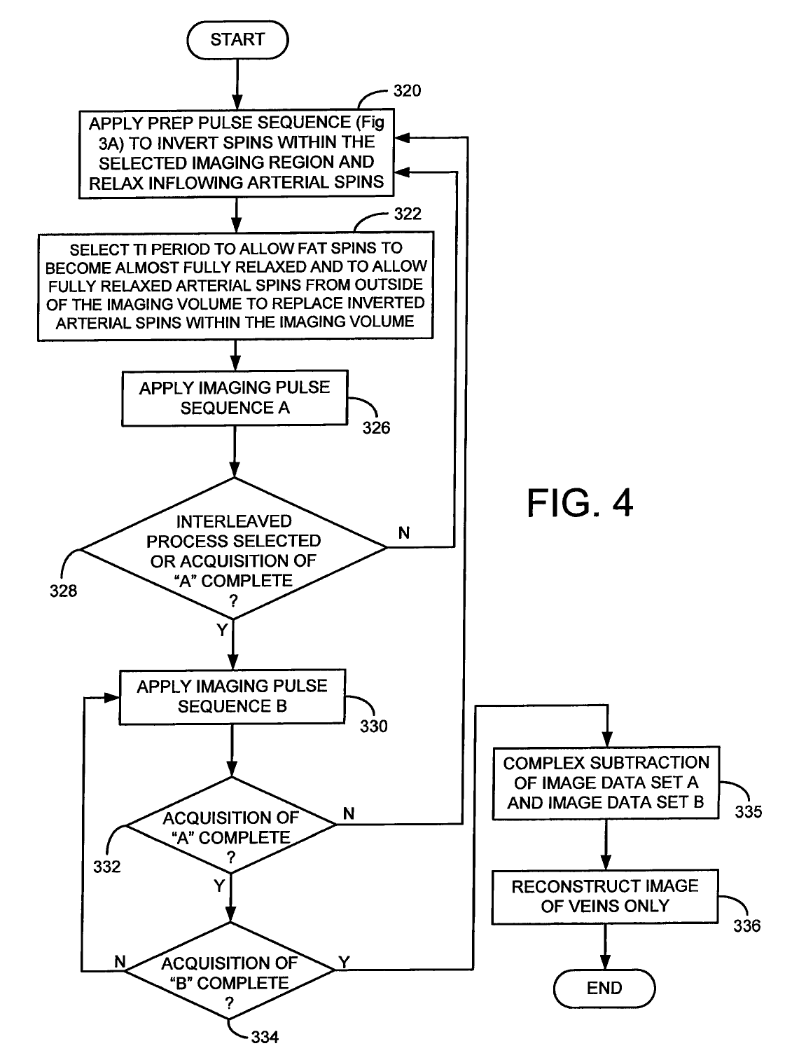

The present invention acquires and utilizes two data sets, hereinafter referred to as image set A and image set B. Referring now to FIG. 3, a general pulse sequence in accordance with the present invention includes preparatory (prep) or tagging pulse sequence 300 and an imaging pulse sequence 302. The prep pulse sequence 300 contains at least one RF pulse and may contain magnetic field gradients used for slice selection or dephasing of transverse magnetization. As will be described below with respect to FIGS. 4-6, the general pulse sequence illustrated in FIG. 3 may be modified for particular imaging processes, such as arterial images, venous images, and the like.

Continuing with respect to FIG. 3, for image set A, the prep pulse sequence 300 is applied to spins within a selected region so as to substantially reduce their longitudinal magnetization, followed by the imaging pulse sequence 302. For example, the prep pulse sequence used to acquire image set A may begin with a RF pulse 308 and an optional associated gradient 309. Thus, the gradient may or may not have an amplitude of zero. Depending on circumstances, the prep pulse sequence 300 may contain one or more RF pulses of that can be spatially selective (i.e. applied to a selected region of interest from which image data is acquired) or non-selective (i.e. applied to a selected region along with all adjacent regions). In general, the RF pulses within the prep pulse sequence 300 should use flip angles of approximately 180 degrees, although in certain circumstances larger or smaller flip angles might be desirable.

Following a recovery time (TI) 310 that is relatively long, for example, at least equal to the T₁ relaxation time of fat and, preferably, at least three times the T₁ relaxation time of fat, data is acquired using a two-dimensional (2D) or three-dimensional (3D) imaging pulse sequence 312. The imaging pulse sequence used for data collection is preferably a balanced steady-state free precession (SSFP) pulse sequence (also known as trueFISP or FIESTA). The use of balanced SSFP pulse sequence is particularly helpful for venous imaging, since the technique naturally makes the blood vessels appear bright. In certain circumstances, however, alternative pulse sequences, such as spoiled gradient-echo or turbo spin-echo, may be preferable. A delay time (TD) 311 may succeed the imaging pulse sequence 312. For image set B, data is acquired in an identical manner, except that no preparatory pulse is applied. If delay times 311 are used, delay times for image sets A and B may or may not be equivalent.

These pulse sequences are repeated as necessary to acquire first and second image data sets A and B from which images can be reconstructed. Image sets A and B are subtracted to identify signal differences between images. The subtraction process is preferably performed in the complex rather than in the magnitude image domain, since artifacts are minimized and vessel contrast is maximized. However, in certain circumstances a magnitude subtraction may be preferred.

The prep pulse sequence 300 reduces the signal intensity of both arteries and veins, which have moderately long T₁ relaxation times. On the other hand, fat has a short T₁ relaxation time so that its longitudinal magnetization almost completely recovers from the effect of the prep pulse sequence 300 during the TI period. Thus, the fat NMR signals are substantially the same in both image data sets A and B and the signals from fat spins are suppressed upon subtraction. When an SSFP imaging sequence is used, muscle, tendons, and ligaments appear dark in both image sets, so that their signals are also suppressed upon subtraction. It is noted that the same would be true if a turbo spin-echo imaging sequence were used for data acquisition.

Blood vessels appear bright in the subtracted images because the signal intensity of the blood in the vessels is larger in image set B (no prep) than in image set A, resulting in a large signal difference upon subtraction. As will be described, depending on the choice of imaging parameters, MR angiograms can be created using the pulse sequence of FIG. 3 that display both arteries and veins, veins alone, or arteries alone.

The invention may be employed to image veins only by judicious use of the prep pulse sequence 300 as shown in FIG. 3A. Referring to FIG. 4 and FIG. 3A, at process block 320 for pulse sequence A, prep pulse 304 and associated gradient 309a are applied to selectively invert inflowing arterial spins located outside of the selected imaging region, then prep pulse 308 (and optional gradient 309b) is applied to all spins. Whereas spins within the selected imaging region are inverted by prep pulse 308, the inflowing arterial spins are re-inverted to become fully relaxed. A TI period is then played out at process block 322. The TI period is sufficiently long so that fat spins recover and so that arterial spins within the selected imaging region flow out of the region and are replaced by fresh, fully magnetized inflowing arterial blood spins. The imaging pulse sequence for image data set A is then applied at process block 326. Therefore, the data acquired using the imaging pulse sequence for image data set A includes fresh arterial spins. As will be described, these fresh arterial spins produce substantially the same NMR signal intensity for image set A as the arterial spins produce for image set B. If a turbo spin-echo readout is used, the prep pulse 304 and associated gradient 309a may not be needed since fast arterial flow can be suppressed on both image sets A and B, leading to suppression of arterial spins and enhancement of venous spins with image subtraction.

The acquisition of image data set A and image data set B can be performed in a sequential or interleaved manner. As will be described, sequential acquisition is advantageous for the removal of fluids, such as joint fluid, from the resulting images. On the other hand, an interleaved acquisition may be advantageously utilized to reduce motion artifacts.

If a sequential process was selected and is not yet complete at decision block 328, the preceding steps are repeated to acquire all data for image data set A. If an interleaved process was selected or image data set A is complete at decision block 328, then an imaging pulse sequence for imaging data set B is applied at process block 330.

If an interleaved process was selected and acquisition of image data set A is not yet complete at decision block 332, each time the preceding steps are performed, an imaging pulse sequence for image data set B is applied at process block 330 and is followed by the pulse sequence for image data set A. However, if a sequential process was selected, image data set A will be complete at decision block 332 and image data set B will be incomplete at decision block 334. Accordingly, imaging pulse sequence B will be repeatedly applied at process block 330 until all of image data set B is acquired. Once image data sets A and B are complete, corresponding views in image data sets A and B are subtracted from each other using complex subtraction, as indicated at process block 335. However, in certain circumstances a magnitude subtraction may be preferred.

The fresh arterial spins included in image data set A produce substantially similar signal intensity for image data set A as the arterial spins for image set B. The arterial signals are, therefore, suppressed upon subtraction at process block 335 and an image in which the veins and not the arteries have contrast will be produced in the difference image reconstructed at process block 336.

It should be noted that a substantially similar result could be obtained by applying the prep pulse 304 at the beginning of pulse sequence B rather than pulse sequence A.

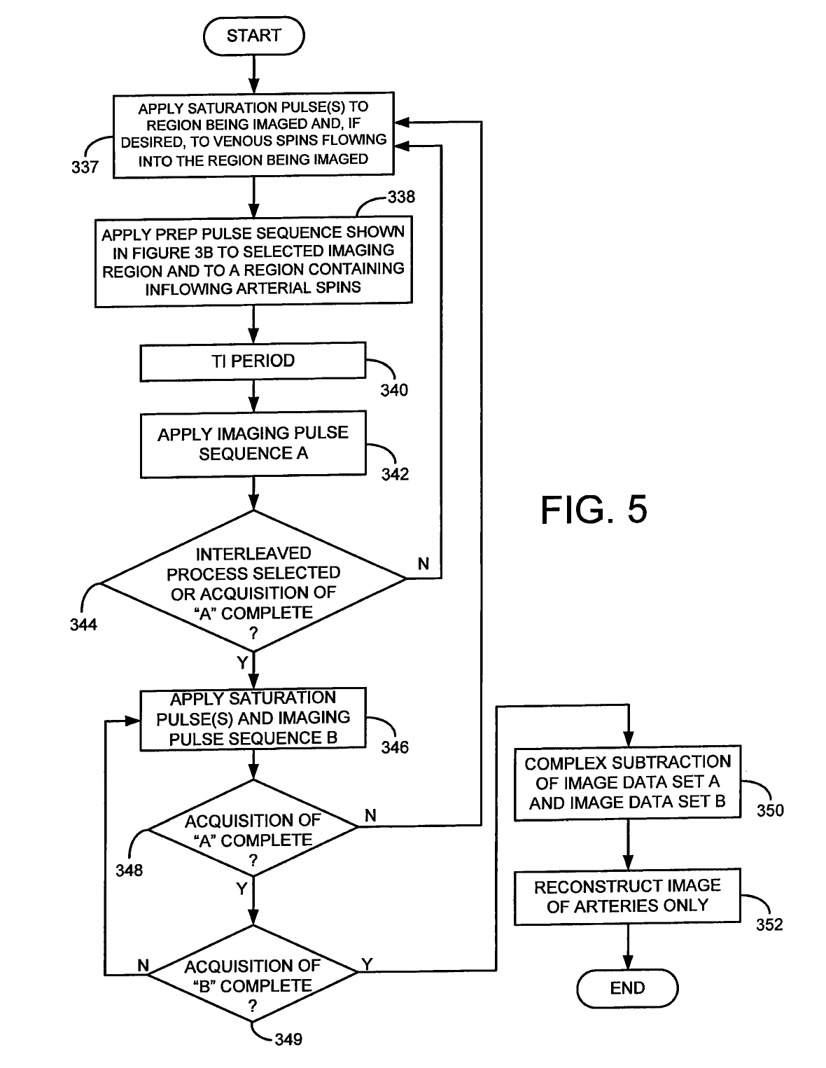

Referring now to FIGS. 5 and 3B, in order to image arteries only, the prep pulse sequence 300 is employed in different manner as shown in FIG. 3B. At process block 337, one or more saturation pulse is applied to the region of interest being imaged and/or to regions surrounding the region of interest being imaged to suppress venous signals. As indicated at process block 338, the prep pulse sequence 300 for image set A is applied to invert longitudinal magnetization in the selected region of interest to be imaged, as well as to the adjacent region from which inflowing arterial spins arrive. At process block 340, during the subsequent TI period, some arterial spins within the selected region may flow out of the region; however, these spins are replaced by the inflow of arterial spins that have experienced the same prep pulse sequence. The arterial blood signal, thus, remains suppressed during the application of the imaging pulse sequence for image data set A at process block 342. As a result of the saturation pulses applied at process block 337, the venous spins for both image data sets A and B produce a relatively low NMR signal level at process block 342.

The process continues at decision block 344 where, as described above with respect to FIG. 4, the process flow may vary depending on the choice of a sequential or interleaved acquisition process. In either case, at process block 346 the imaging pulse sequence is applied to acquire image data set B. In addition to the imaging pulse sequence, one or more saturation pulses may be applied as described above to suppress the signal from venous spins residing in the region to be imaged and/or to venous spins flowing into the region to be imaged. Once image data sets A and B are determined to be complete at decision blocks 348 and 349, the data sets are subtracted using complex subtraction as described above at process block 350. However, in certain circumstances a magnitude subtraction may be preferred.

With this embodiment, arterial spins within the selected region of interest may flow out of the region during the acquisition of data set A; however, these spins are replaced by the inflow of arterial spins that have experienced the same prep pulse sequence 300, so the arterial signal remains suppressed. On the other hand, for image set B, these spins are replaced by the inflow of fully magnetized arterial spins that produce high signal. Accordingly, when image data set A and B are subtracted at process block 350, only the arterial spins are depicted, without signal contributions from fat or muscle. In addition, since one or more saturation pulses were applied to resident or inflowing venous spins for both image sets A and B, these saturated spins result in the veins having a comparably low signal in both image sets. Therefore, the venous signal is suppressed upon image subtraction at process block 350 and an image of the arteries alone can be reconstructed at process block 352. In addition, it is contemplated that the venous signal can also be reduced by the application of a T₂-preparation for both image sets.

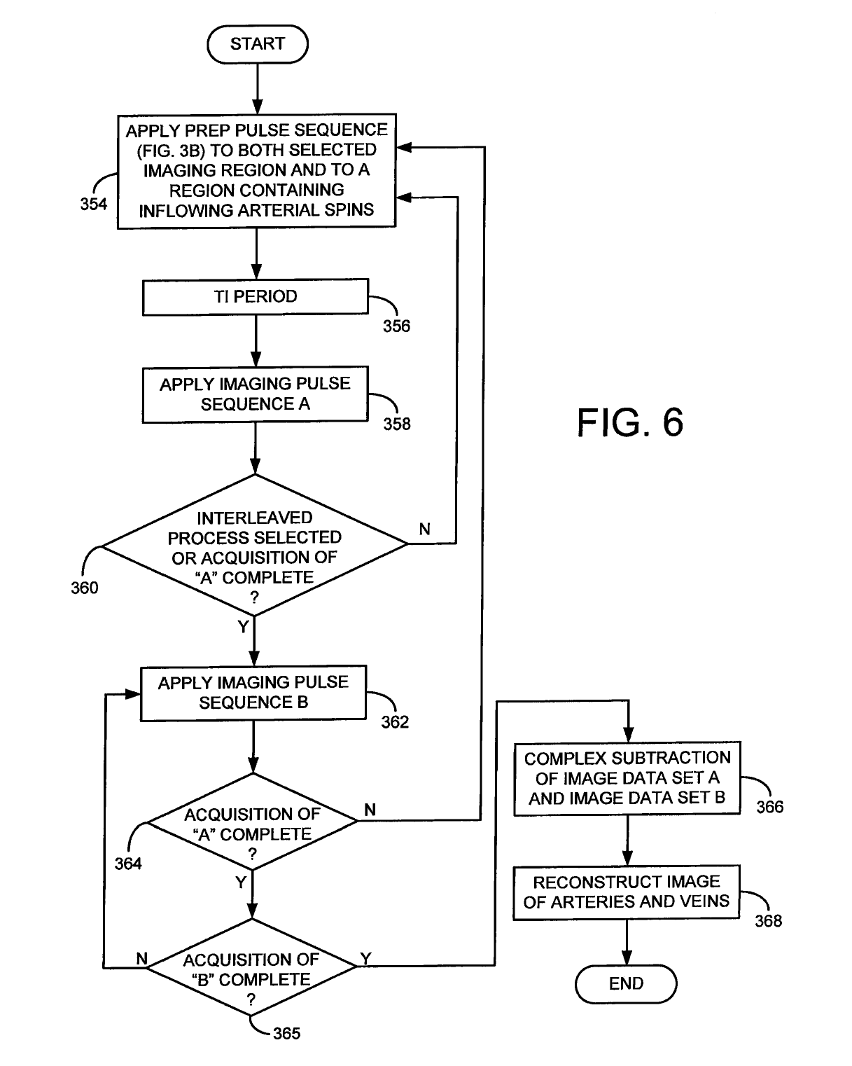

Referring now to FIGS. 6 and 3B, in order to image both arteries and veins, the prep pulse sequence 300 as shown in FIG. 3B for image data set A is applied to both the selected region of interest to be imaged as well as to inflowing arterial spins at process block 354. However, unlike the artery-only case described above with respect to FIG. 5, no saturation pulses are applied so that the veins remain visible after subtraction. That is, at process block 354, the prep RF pulse shown in FIG. 3B is applied to both the selected imaging region and to a region containing inflowing arterial spins.

At process block 356, during the subsequent TI period, some arterial spins within the selected region may flow out of the region. However, these spins are replaced by the inflow of arterial spins that have experienced the same prep pulse sequence, so the arterial signal remains suppressed during the application of the imaging pulse sequence for image data set A at process block 358.

The process continues at decision block 360 where, as described above with respect to FIG. 4, the process flow may vary depending on the choice of a sequential or interleaved acquisition process. In either case, at process block 362 the imaging pulse sequence is applied to acquire image data set B and, once image data sets A and B are determined to be complete at decision blocks 364 and 365, the data sets are subtracted using complex subtraction at process block 366. However, in certain circumstances a magnitude subtraction may be preferred.

As described above, during acquisition of imaging data for image data set A, arterial spins within the selected region may flow out of the region; however, these spins are replaced by the inflow of arterial spins that have experienced the same prep pulse sequence, so the arterial signal remains suppressed. On the other hand, for image data set B, these spins are replaced by the inflow of fully magnetized arterial spins that produce high signal. Accordingly, when image data set A and B are subtracted at process block 366, the arterial spins are depicted, without signal contributions from fat or muscle. In addition, since no saturation pulses were applied to inflowing or resident venous spins for image set A, the venous signal is not suppressed upon image subtraction at process block 366 and an image of the arteries and veins can be reconstructed at process block 368.

It should be noted that fluids such as joint effusions may appear bright with the above-described STARFIRE technique. However, there are several methods that can be applied in conjunction with the STARFIRE technique to reduce fluid signal while maintaining intravascular signal. For instance, a threshold can be set to suppress data based on the signal intensity ratio of the two image sets, since fluids will have a distinctly different ratio from other tissues. Alternatively, fluids can be distinguished and suppressed based on their longer T₁ relaxation time.

For the purpose of removing unwanted fluid signal, a sequential (non-interleaved) STARFIRE acquisition can be used that seeks to null fluid signal on the RF tagged image set. The acquisition yields high signal from fluids on the untagged image set (image data set B) and allows for removal of fluid voxels in the subtracted STARFIRE angiographic image based their signal ratio on the untagged to RF tagged images. Examples of such signal ratios are:

$$R = \frac{{S}_{U}}{{S}_{T}} and R = \frac{{S}_{U}^{2}}{[ {S}_{T} \cdot max ( {S}_{U} ) ]} ;$$

where S_{U} and S_{T} denote voxel intensities in the untagged (image data set B) and tagged (image data set A), respectively, and max(S_{U}) denotes the maximum voxel intensity in the untagged image set B. Image voxels with appreciably large values of R would be attributable to fluids and could, with user interaction or with fully automated computer assisted strategies, be removed from of the angiographic STARFIRE image sets.

An alternative method for removing unwanted fluid signal involves the use of two acquisitions for both of which an inversion preparation is applied. The repetition time and TI for each acquisition are selected so that the fluid signal and background signals are similar while the intravascular signal is substantially different. After image subtraction, the fluid signal and background signal is minimized while the intravascular signal is preserved.

The above-described methods do not require the administration of a contrast agent in order to create an MR angiogram. Unlike time of flight methods, STARFIRE is not sensitive to the direction or velocity of flow. Also, unlike phase contrast angiography, it does not require prior knowledge of the velocity of flow. Moreover, the image quality is far superior to that provided by other non-contrast methods. Also, unlike previously described non-contrast methods, STARFIRE allows selective imaging of arteries or veins.

It is contemplated that the first pulse sequence A and the second pulse sequence B can be utilized together to acquire accurate images in the presence of a highly homogenous static magnetic field or in the presence of a static magnetic field that is inhomogeneous. As will now be described, in order to take advantage of the respective strengths of the GRE and SSFP techniques, while minimizing their disadvantages, MR angiographic images are acquired using the present invention in the presence of an inhomogeneous magnetic field using the GRE method, whereas an SSFP pulse sequence is employed where the magnetic field is homogeneous.

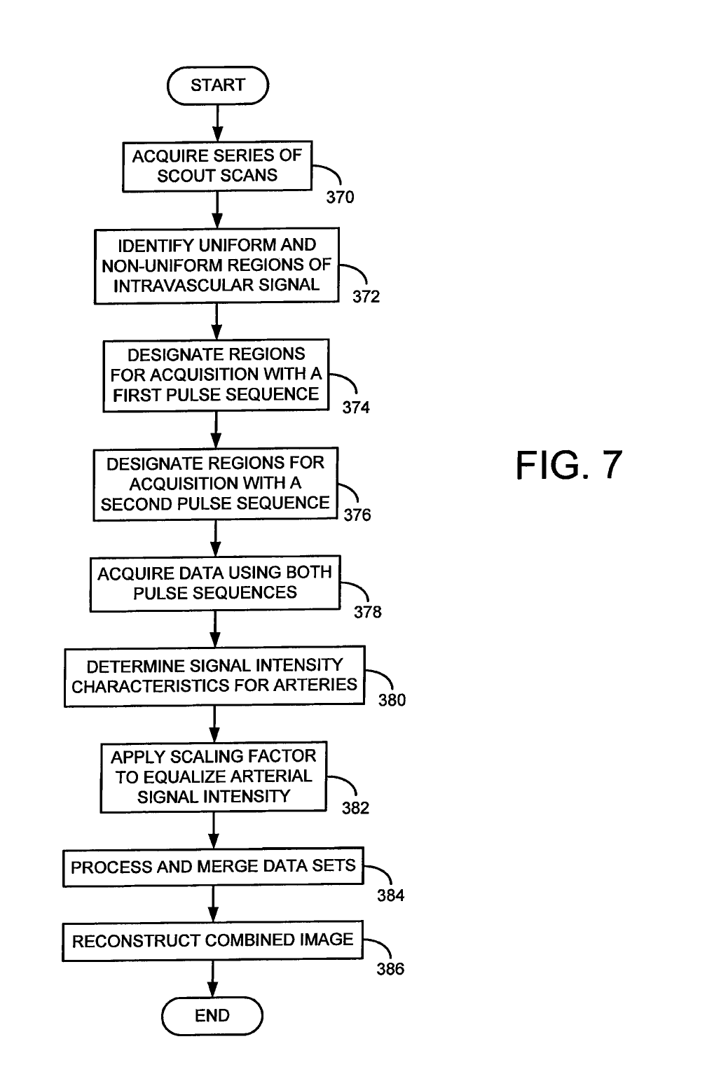

Referring now to FIG. 7, a process for imaging vasculature starts at process block 370 by acquiring a series of scout images through the entire region of blood circulation using an ultra-fast pulse sequence sensitive to magnetic field imperfections (for instance, SSFP). Thereafter, at process block 372, the regions of uniform and non-uniform intravascular signal are identified. This step of uniform and non-uniform intravascular signal may be performed manually by reviewing the scout scans acquired at process block 370. On the other hand, it is contemplated that a threshold for acceptable local magnetic field homogeneity may be used. This threshold can be based in whole or part on various factors including, for example, characteristics of the pulse sequences and MR scanner, physical characteristics of the body part being imaged, anatomic and flow characteristics of the targeted vascular territory, direct measurement of magnetic field homogeneity using field mapping techniques, or the detection of magnetic field-sensitive flow artifacts in the images. In this case, regions in which the local magnetic field homogeneity is beneath the threshold level are designated for acquisition using a first pulse sequence. Similarly, regions in which the local magnetic field homogeneity is above the threshold level are designated for acquisition with the second pulse sequence.

From this review, the regions in which the intravascular signal appears non-uniform are designated for acquisition using the first pulse sequence at process block 374 and the regions in which the intravascular signal appears uniform are designated for acquisition using the second pulse sequence at process block 376. At process block 378, data is acquired using the first pulse sequence and the second pulse sequence. The data acquisition at process block 378 is performed such that there is partial overlap of the regions imaged with the first pulse sequence and the second pulse sequence.

After data acquisition is complete, the signal intensity characteristics for arteries are determined in the data acquired using the first pulse sequence and the second pulse sequence at process block 380. A scaling factor is applied to the data at process block 382. Preferably, the scaling factor is applied to the GRE image data acquired, so both image data sets have comparable levels of arterial signal intensity. In general, the two acquisitions preferably use comparable spatial resolution (e.g. slice thickness and field of view), although this is not required. The two image data sets are aligned, corrected for geometrical distortion, and merged into a single data set at process block 384. In particular, the images are processed into an angiogram using a maximum intensity projection or volume rendering technique and reconstructed into a combined image at process block 386.

The above-described method can be implemented using numerous variations of 3D GRE and 3D SSFP acquisition methods. For instance, the STAR method of arterial spin labeling can be used to create MR angiograms in which the background signal intensity is suppressed. In the intracranial circulation for example, a 3D STAR GRE pulse sequence is acquired through the region of the circle of Willis, which is in close proximity to the skull base (where the static magnetic field is inhomogeneous). The remainder of the intracranial circulation is imaged using a 3D STAR SSFP pulse sequence in order to maximize vessel SNR, as well as to minimize saturation effects in the slower flowing intravascular spins within the more distal vessels.

This “hybrid STAR” technique provides significantly improved depiction of the intracranial circulation than has previously been feasible using 3D TOF or contrast-enhanced approaches. The technique yields a high degree of background suppression and consequently large vessel-to-background contrast. The hybrid STAR technique avoids the severe disruption of intravascular signal that would otherwise occur with true FISP for the portions of the circulation where substantial static magnetic field inhomogeneities are present. Moreover, no cardiac gating is required. The technique may be further improved by using 2D parallel imaging techniques to further reduce scan time and phased array coils with larger numbers of elements to improve SNR.

It should also be noted that the above-described principles can be further modified to perform multi-phase, two-dimensional (2D) or three-dimensional (3D) time-of-flight MR cineangiography. In a manner similar to that described above, the method provides for substantially complete background signal suppression and high temporal resolution using a multi-phase, undersampled acquisition synchronized to the cardiac cycle. High temporal resolution can be achieved by the use of k-space undersampling, preferably with radial k-space trajectories. Undersampling artifacts can be eliminated by the subtraction of the two data sets.

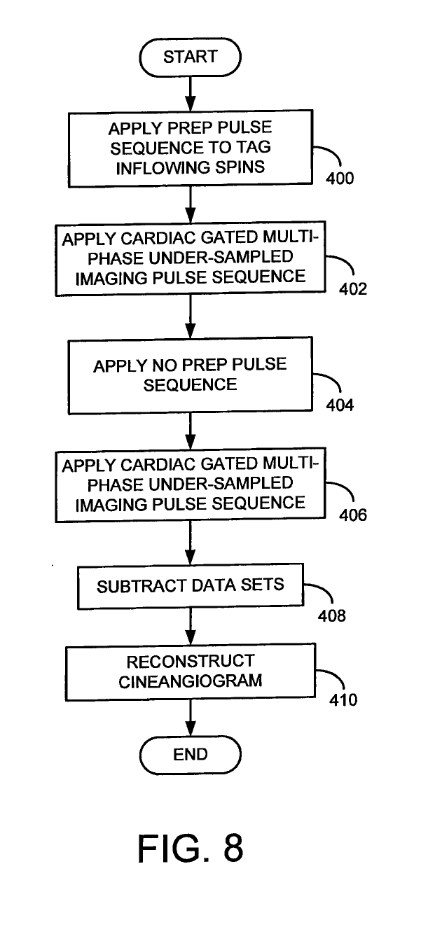

Specifically, referring to FIG. 8, in order to reduce or substantially eliminate undersampling artifacts, two image sets are acquired. In a manner similar to that described above, the first of the two image sets is acquired after a prep pulse sequence that tags the magnetization of the inflowing spins at process block 400. Following thereafter, at process block 402, the first cineangiographic data set is acquired using a multi-phase, undersampled acquisition synchronized to the cardiac cycle. After the first image set is acquired, no prep pulse sequence is applied as indicated by process block 404. Following thereafter, at process block 406, the second cineangiographic data set is acquired with a multiphase, undersampled acquisition synchronized to the cardiac cycle. The two image sets are then subtracted at process block 408 and a cineangiogram is reconstructed at process block 410 in which the motion of the tagged blood (which appears bright against a background of negligible signal intensity) is highly visible as it passes through the imaging volume.

Therefore, the above-described method obtains time-of-flight, cineangiograms that display vascular anatomy and flow patterns with high vessel conspicuity, high temporal resolution, and minimal artifacts. Temporal resolution can be increased arbitrarily by varying the undersampling factor and/or by using parallel imaging with high acceleration factors.

The method enables the creation of time-resolved MR angiograms with temporal resolution on the order of tens or hundreds of milliseconds. This level of temporal resolution is beyond the capability of contrast-enhanced MRA methods; moreover, there is no need for a contrast agent. Unlike time of flight methods, it is not sensitive to the direction or velocity of flow. Unlike phase contrast angiography, it does not require prior knowledge of the velocity of flow. Furthermore, the image quality is far superior to that provided by other non-contrast methods.

The present invention has been described in terms of the various embodiments, and it should be appreciated that many equivalents, alternatives, variations, and modifications, aside from those expressly stated, are possible and within the scope of the invention. Therefore, the invention should not be limited to a particular described embodiment.

1. A method for producing an angiogram with a magnetic resonance imaging (MRI) system, the method comprising the steps of:
a) performing a preparatory pulse sequence that includes application of an RF pulse that reduces longitudinal magnetization of spins in a region of interest;
b) performing an image acquisition pulse sequence following a time interval (TI) after step a) to acquire complex image data in which the NMR signals from blood is suppressed, the NMR signals from fat are substantially recovered, and the NMR signals from other tissues are reduced;
c) repeating the image acquisition pulse sequence following step b) to acquire complex image data in which the NMR signals from blood is recovered, the NMR signals from fat are substantially recovered, and the NMR signals from other tissues are reduced;
d) repeating steps a), b), and c) to form a first complex image data set from the complex image data acquired in step b) and a second complex image data set from the complex image data acquired in step c); and
e) performing a subtraction of the first and second image data sets to produce an angiogram in which blood vessels have an enhanced brightness.

2. The method of claim 1 wherein the image acquisition pulse sequence is at least one of a steady-state free precision (SSFP) pulse sequence and turbo spin-echo pulse sequence that produces images with bright blood vessels.

3. The method of claim 1 wherein the subtraction in step e) includes at least one of complex and magnitude subtraction.

4. The method of claim 1 wherein step b) the TI is greater than a relaxation time of background structures in the region of interest.

5. The method of claim 4 wherein the TI is at least three times a T₁ relaxation time of fat.

6. The method of claim 1 wherein the repeated image acquisition pulse sequence also includes application of an RF pulse that reduces longitudinal magnetization of spins in a region of interest, but uses at least one of a different repetition time and TI from the image acquisition pulse sequence such that fluid and background signals are similar on both acquisitions, but intravascular signal is substantially different.

7. The method of claim 1 further comprising designating a first region of interest and a second region of interest and performing steps a)-e) with respect to the first region of interest using a first image acquisition pulse sequence and performing steps a)-e) with respect to the second region of interest using a second image acquisition pulse sequence.

8. The method of claim 7 wherein the first region of interest includes structures that induce inhomogeneities in the static magnetic field and the second region of interest is substantially free of structures that induce inhomogeneities in the static magnetic field.

9. The method of claim 8 wherein the first image acquisition pulse sequence includes a GRE pulse sequence and the second image acquisition pulse sequence includes a SSFP pulse sequence.

10. The method of claim 7 wherein an undersampled data acquisition is applied and image subtraction is used to suppress undersampling artifacts.
